(19) United States
(12) Patent Application Publication
(10) Pub. No.: US 2009/0112208 A1
(43) Pub. Date: Apr. 30, 2009

(54) EXTERNAL BONE SCREW SYSTEM AND METHOD OF USE FOR FRACTURES, FUSIONS OR OSTEOTOMIES

(76) Inventors: Anthony V. Borgia, Las Vegas, NV (US); Axel A. Weller, Las Vegas, NV (US)

Correspondence Address: GREENBERG TRAURIG, 3773 HOWARD HUGHES PARKWAY, SUITE 500 NORTH, LAS VEGAS, NV, 89169, US

(21) Appl. No.: 11/924,566

(22) Filed: Oct. 25, 2007

(51) Int. Cl. A61B 17/00; A61B 17/04; A61B 17/58

(52) U.S. Cl. 606/59; 606/301; 606/104

(57) ABSTRACT

A bone screw system is disclosed. The bone screw system allows a bone screw to be inserted into a bone via a guide set with a plurality of pins. After the bone screw is inserted, a head portion of an extension member attached to the bone screw remains external to the wound such that the bone screw can be tightened later without re-opening the wound. The bone screw system is applicable to bone fractures, bone fusions, Osteomonies and other bone connectivity procedures. After healing, the bone screw may be left in the patient by removing an upper portion or the screw may be removed with additional incisions and a local anesthetic.

### FIELD OF THE INVENTION

The embodiments of the present invention relate to a bone screw system for helping bone fractures, fusions or Ostetomnies to mend. More particularly, the embodiments relate to a bone screw system comprising a bone screw and extension member having a portion which remains external to a patient's skin allowing the screw to be tightened without accessing the fracture, fusion or Osteotomy via an incision or other invasive procedure. The screw can be left in by detaching external components after healing or the screw may be removed in its entirety.

### BACKGROUND

The medical industry has no shortage of bone screws. Bone screws are implanted into patients to immobilize all types of bones fractures and fusions. Unfortunately, as patients undertake daily activities, the bone screws tend to loosen requiring them to be tightened. Tightening an implanted bone screw requires the bone fracture or fusion area to be accessed. In most instances, an incision is made, the bone screw is tightened and the incision is closed via stitches or a butterfly suture. Besides being stressful for patients, the incision procedure comes with risk. Infection and other side effects of the incision procedure make the procedure less than routine.

Thus, there exists a need for a bone screw system that functions as well as implanted bone screws but allows bone screws to be tightened without any secondary invasive procedure on the patient.

### SUMMARY

Accordingly, one system embodiment of the present invention comprises: an elongated guide defining a passageway; and a bone screw having a threaded first end for insertion into bone and a second end adapted to engage a first end of a hollow extension member wherein a second end of said hollow extension member is adapted to remain external to a wound after insertion of the bone screw into the bone.

Another system embodiment comprises: an elongated circular guide defining a passageway, said guide having multiple channels from a first end to a second end of a guide wall; guide pins adapted to extend along the channels, said pins having heads at a first end that prevent the guide pins from passing through the channels and second ends extending from said channels adapted to set the guide to bone; and a bone screw having a threaded first end for insertion into said bone and a second end adapted to engage a first end of a hollow extension member, a second end of said hollow extension member adapted to remain external to a wound and engage a tightening tool for tightening the bone screw.

A first method embodiment of the present invention comprises: making an incision near bone; inserting a guide into said incision such that said guide is adjacent to the bone; setting said guide to the bone; drilling a cavity into said bone via a passageway in the guide; inserting a bone screw via said passageway into said bone such that one end of an attached hollow extension member remains external to the incision, said hollow extension member adapted to allow a tightening device to pass therethrough to engage said bone screw.

In general, the bone screw of the present invention allows a bone screw to be inserted into a bone via the set guide. After the procedure is completed, a head portion of an extension member remains external to the wound such that the bone screw can be tightened later without re-opening the wound.

Other variations, embodiments and features of the present invention will become evident from the following detailed description, drawings and claims.

### BRIEF DESCRIPTION OF THE DRAWINGS

FIG. 1 illustrates an exploded view of various components of one bone screw system embodiment of the present invention;

FIG. 1a illustrates an end view of a bone screw guide;

FIG. 2 illustrates a perspective view of a bone screw, tightening device and plug;

FIG. 3 illustrates a side view of the bone screw, tightening device and plug in a connected configuration;

FIG. 4 illustrates a cross-sectional view along A of the bone screw, tightening device and plug in a connected configuration;

FIG. 5 illustrates a side view of a head portion of the bone screw;

FIG. 6 illustrates a top view of the head portion of the bone screw;

FIG. 7 illustrates a side view of a threaded portion of the bone screw;

FIG. 8 illustrates a perspective view of a countersink tool for countersinking a drilled cavity in a bone;

FIG. 9 illustrates a side view of the countersink tool;

FIG. 10 illustrates a top view of the tool for countersinking the cavity in the bone; and

FIG. 11 illustrates a flow chart detailing one method of using one embodiment of the bone screw system of the present invention.

### DETAILED DESCRIPTION

It will be appreciated by those of ordinary skill in the art that the invention can be embodied in other specific forms without departing from the spirit or essential character thereof. The presently disclosed embodiments are therefore considered in all respects to be illustrative and not restrictive.

While bone fractures are focused on below, bone fusions, Osteotomies and other bone connectivity procedures may benefit from the embodiments of the present invention as well.

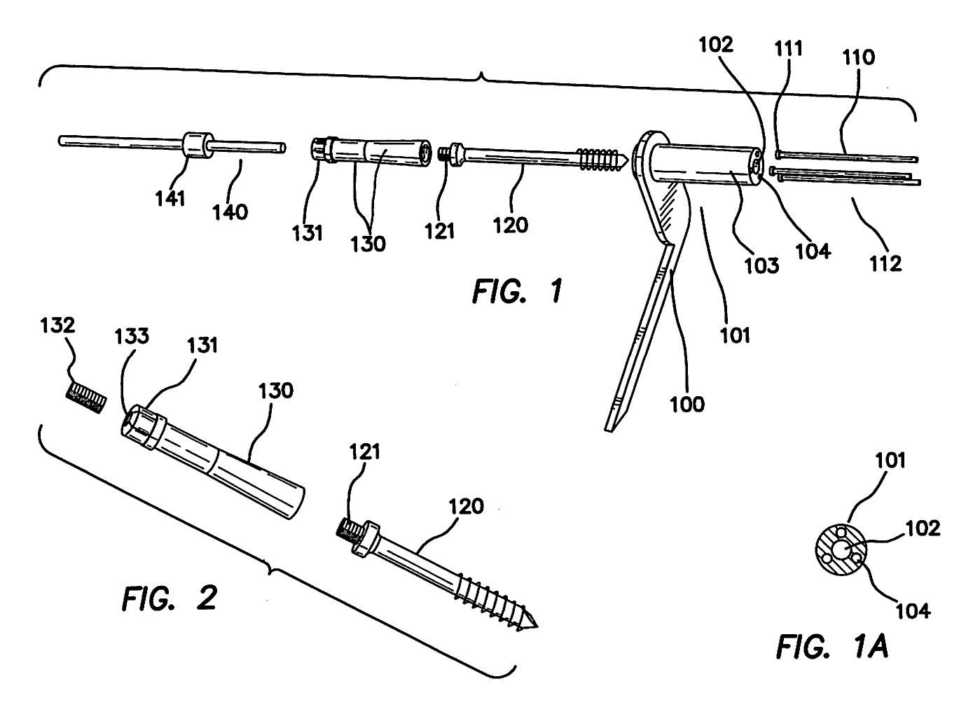

Initial reference is made to FIGS. 1 and 2 illustrating an exploded view of components of one embodiment of a bone screw system of the present invention comprising a guide 100, guide pins 110, bone screw 120, bone screw extension member 130 and bone drill bit 140. FIG. 1a shows an end view of the guide 100.

The guide 100 includes an elongated tube 101 defining a passageway 102 for allowing the bone screw 120 to insert into an incision and a handle 102. A wall 103 of the elongated tube 101 includes, as shown, three channels 104 extending along a length of the tube 101. In practice, after an incision is made near a bone fracture, the tube 101 is inserted into the incision so that it contacts a bone near the fracture. Then, as shown, three guide pins 110 are inserted into the channels 104 and are tapped so that sharpened ends 112 of the pins 110 enter the bone to temporarily set the guide 100 in place. Pin heads 111 prevent the pins 110 from passing completely through the channels 104. Once the guide 100 is set, the bone drill bit 140 is used with a power drill to drill a starter cavity for entry of the bone screw 120.

The bone drill bit 140 also includes a bushing 141 designed to control the depth at which the drill enters the bone. Once the desired depth of drilling is known, the bushing 141 can be positioned accordingly to prevent inadvertent over-drilling.

Once the cavity is drilled, the bone screw 120 is inserted into the passageway 102 and drilled through the fractured bone to secure two bone fragments together so that they may heal properly. However, it is well known that bone screws tend to loosen over time as the patient moves around and otherwise puts external forces on the fracture area. Therefore, tightening a bone screw requires a subsequent incision such that a tightening device can engage and tighten the bone screw. With the embodiments of the present invention, the length of the bone screw 120 and hollow extension member 130 is selected so that a head portion 131 of the extension member 130 remains external to the initial incision.

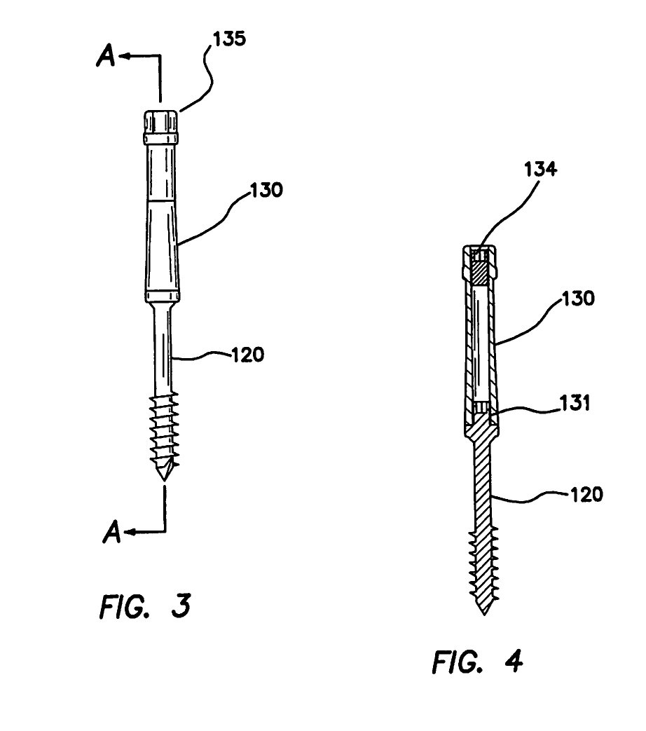

FIGS. 2 through 4 show various views of the bone screw 120 and hollow extension member 130 separated and connected. The extension member 130 includes a threaded internal portion 131 which engages a threaded portion 121 of the bone screw 120. The engagement of the bone screw 120 and extension member 130 remains internal to the wound and is therefore sterilized and sealed to prevent bacteria from entering the wound. Additionally, the threaded portion 131 of the extension member 130 and the threaded portion 121 of the bone screw 120 are machined with very accurate tolerances. Adhesive or a similar sealant may also be used to seal the connection between the extension member 130 and bone screw 120. An opposite end of the extension member 130 includes a threaded plug 132 for sealing an opening 133. The plug 132 has a hexagonal cavity 134 for receipt of an Allen wrench or similar tool for tightening the bone screw 120 after insertion. Alternatively, a hexagonal-shaped portion 134 of the extension member 130 allows a socket wrench or similar tool to be affixed temporarily thereto for tightening the bone screw 120 after insertion.

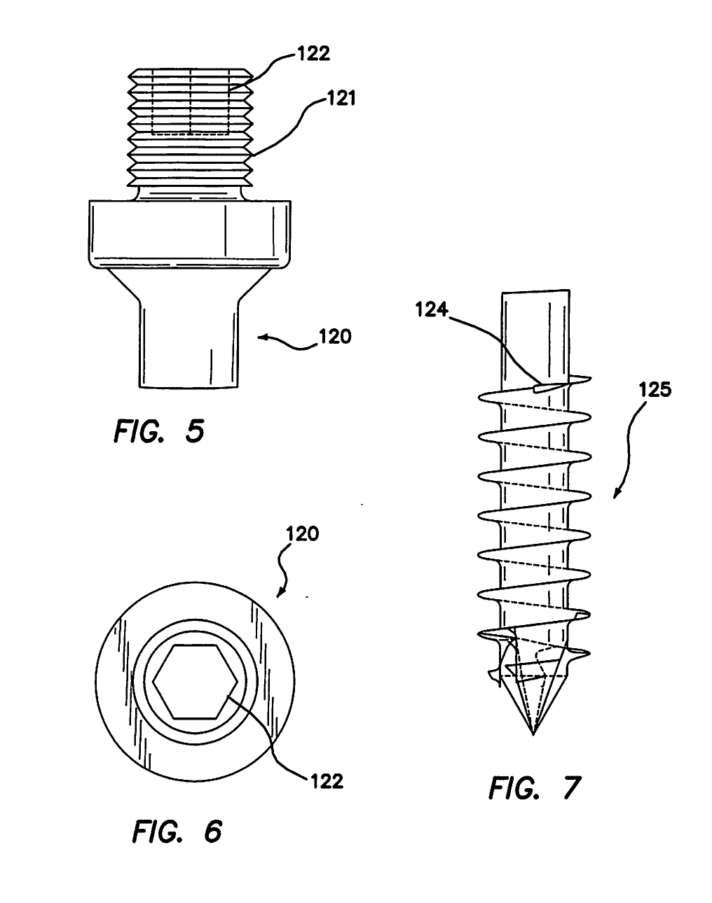

Now referring to FIGS. 5 through 7, the aspects of the bone screw 120 are shown in detail. FIGS. 5 and 6 show an upper threaded portion 121 of the bone screw 120 for engagement with the extension member 130. The threaded portion 121 includes a hexagonal cavity 122 bored or otherwise formed therein. The hexagonal cavity 122 accepts an Allen wrench or similar tool for direct tightening of the bone screw 120. FIG. 7 shows a 60° self-tapping, threaded lower end 125 of the bone screw 120 that is used to penetrate bone. A flat spot 124 on the threaded lower end 125 of the bone screw 120 indicates a termination point of the threads. In one embodiment, adjacent threads are spaced 0.11 inches and the width of the threaded lower end 125 is 0.311 inches. Those skilled in the art will recognize that the bone screw 120 may be any suitable size dependent upon the procedure to be completed.

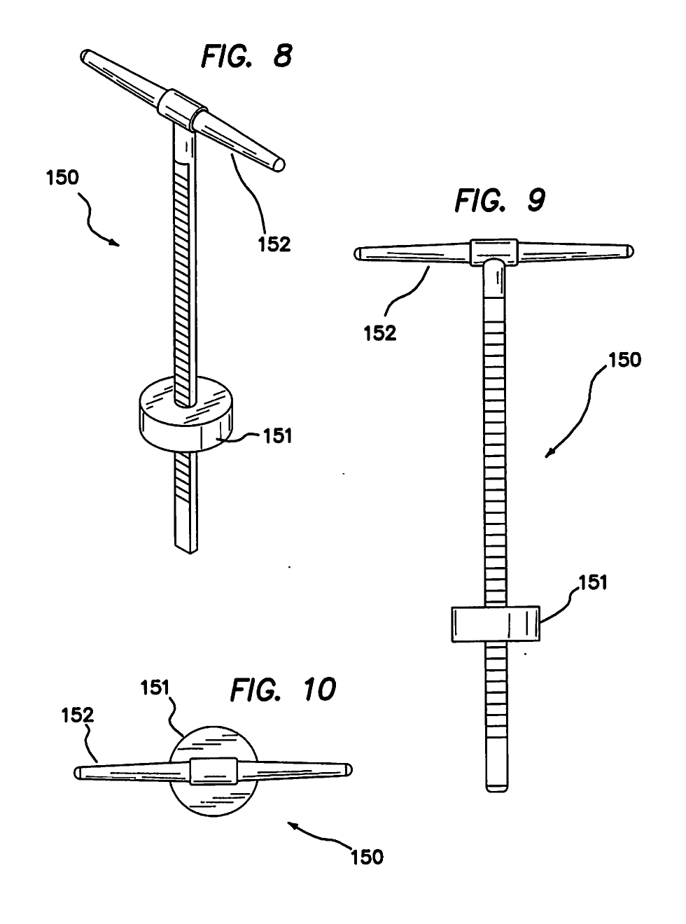

FIGS. 8 through 10 show various views of an optional countersink tool 150 that is used after the bone drill 140 to countersink drilled holes to better accommodate the bone screw 120. The countersink tool 150 also includes a depth bushing 151 to ensure that a resultant countersink does not inadvertently penetrate the bone too deeply. A diameter of the depth bushing 151 should be less than that of the passageway of the guide 100 to permit the countersink tool 150 to be used with the guide 100 in its set position. A handle 152 provides means for creating the necessary torque to countersink bone at the drilled hole locations.

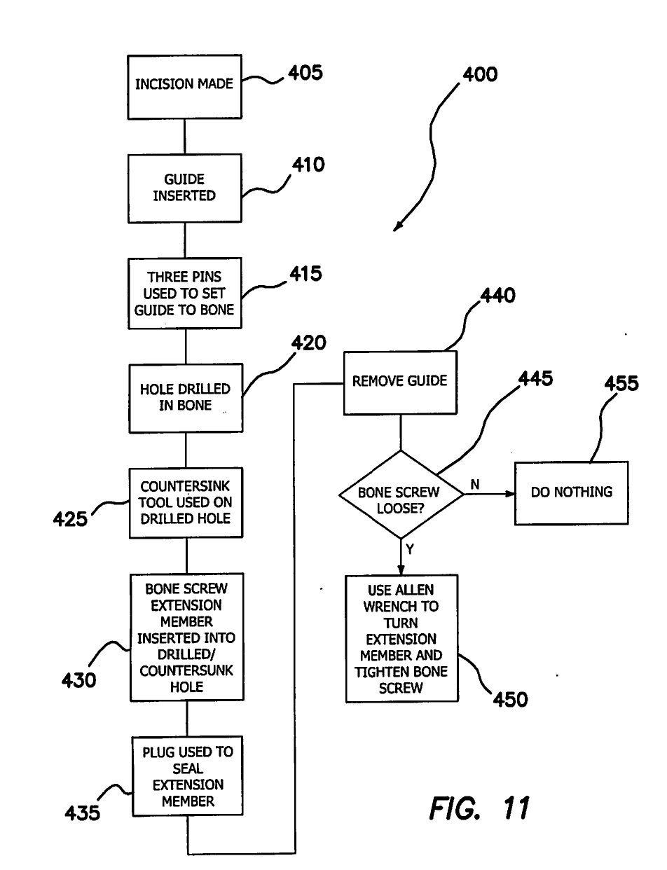

FIG. 11 illustrates a flow chart 400 detailing one method of using the bone screw system of the present invention. At 405, an incision is made in the patient at the location of the bone fracture to be treated. Prior to the incision, the area is also treated with a providone-iodine topical antiseptic (e.g., Betadine®) to reduce the risk of infection. At 410, the guide 100 is inserted into the incision such that the guide passageway rests over the bone fracture area to be treated. A guide handle 102 provides means for inserting the guide 100. At 415, three pins 110 are inserted into respective channels 103 in the guide wall 104 and set into the bone. At 420, drill bit 140 and bushing 141 are used to drill a hole of appropriate depth into the bone. At 425, the countersink tool 150 and depth bushing 151 are used to countersink the drilled hole to an appropriate depth. At 430, the bone screw 120 and attached extension member 130 are inserted into the guide 100 and the bone screw 120 is screwed through the bone fracture such that the two sections of fractured bone are positioned for healing. At 435, the plug 132 is used to seal of the hollow extension member 130. At 440, the guide 100 is removed from the incision. At 445, after a passage of time, and likely at a follow-up status appointment with the surgeon, it is determined whether the bone screw 120 has loosened. If so, at 450, an Allen wrench or similar device is inserted into the hexagonal cavity 134 of plug 132 and turned to tighten the bone screw 120 as desired. If not, at 455, nothing need be done. The embodiments of the present invention eliminate the need to utilize follow-up invasive procedures to tighten bone screws. Therefore, the system of the present invention reduces patient stress associated with follow-up invasive procedures and reduces the risk of complications associated therewith.

In addition, after healing, the screw can be left in by detaching an upper portion of the screw or the entire system may be removed. The upper portion may be removable (e.g., threaded) or cut off using appropriate tooling.

Each of the components of each embodiment of the bone screw system of the present invention maybe fabricated of any suitable materials including metal alloys and composites. Ideally, the manufacturing process utilizes small tolerances to ensure the preciseness and operability of the system.

Although the invention has been described in detail with reference to several embodiments, additional variations and modifications exist within the scope and spirit of the invention as described and defined in the following claims.

We claim:

1. A bone screw system comprising:
an elongated guide defining a passageway; and
a bone screw having a threaded first end for insertion into bone and a second end adapted to engage a first end of a hollow extension member wherein a second end of said hollow extension member is adapted to remain external to a wound after insertion of the bone screw into the bone.

2. The bone screw system of claim 1 wherein a guide wall includes a series of channels along a length thereof.

3. The bone screw system of claim 2 further comprising a series of pins adapted to insert into said series of channels.

4. The bone screw system of claim 1 wherein the guide includes a handle.

5. The bone screw system of claim 1 wherein the second end of the bone screw is threaded to engage with a threaded first end of the extension member.

6. The bone screw of claim 1 further comprising a threaded plug at a second end of the extension member.

7. The bone screw system of claim 6 wherein the plug includes a hexagonal cavity for engagement with a tightening tool.

8. The bone screw system of claim 1 wherein the second end the bone screw includes a hexagonal cavity.

9. A bone screw system comprising:
an elongated circular guide defining a passageway, said guide having multiple channels from a first end to a second end of a guide wall;
guide pins adapted to extend along the channels, said pins having heads at a first end that prevent the guide pins from passing through the channels and second ends extending from said channels adapted to set the guide to bone; and
a bone screw having a threaded first end for insertion into said bone and a second end adapted to engage a first end of a hollow extension member, a second end of said hollow extension member adapted to remain external to a wound and engage a tightening tool for tightening the bone screw.

10. The bone screw system of claim 9 wherein the guide includes a handle.

11. The bone screw system of claim 9 wherein the second end of the bone screw is threaded to engage with a threaded first end of the extension member.

12. The bone screw system of claim 9 wherein the extension member includes a hexagonal cavity for engagement with a tightening tool.

13. The bone screw system of claim 9 wherein the second end the bone screw includes a hexagonal cavity.

14. A method of using a bone screw system comprising:
making an incision near bone;
inserting a guide into said incision such that said guide is adjacent to the bone;
setting said guide to the bone;
drilling a cavity into said bone via a passageway in the guide; and
inserting a bone screw via said passageway into said bone such that one end of an attached hollow extension member remains external to the incision, said hollow extension member adapted to allow a tightening device to pass therethrough to engage said bone screw.

15. The method of claim 14 further comprising countersinking the drilled cavity in the bone to a pre-established depth prior to inserting the bone screw.

16. The method of claim 14 further comprising using a series of pins to set the guide to the bone.

17. The method of claim 16 further comprising inserting the series of pins through channels in a guide wall.

18. The method of claim 14 further comprising drilling a cavity to a pre-established depth.

19. The method of claim 14 wherein said bone is fractured.

20. The method of claim 14 wherein said bone is to be fused.

21. The method of claim 14 wherein said bone was subjected to a osteotomy procedure.
